(19) United States
(12) Patent Application Publication
(10) Pub. No.: US 2010/0234393 A1
(43) Pub. Date: Sep. 16, 2010

(54) USE OF A VASOPEPTIDASE INHIBITOR FOR THE TREATMENT OF PULMONARY ARTERIAL HYPERTENSION

(76) Inventors: Jean-Charles Schwartz, Paris (FR); Jeanne-Marie Lecomte, Paris (FR)

Correspondence Address: STITES & HARBISON PLLC, 1199 NORTH FAIRFAX STREET, SUITE 900, ALEXANDRIA, VA, 22314, US

(21) Appl. No.: 12/303,981

(22) PCT Filed: Jun. 12, 2007
(86) PCT No.: PCT/FR2007/000970

§ 371 Date: Dec. 9, 2008

(30) Foreign Application Priority Data
Jun. 13, 2006 (FR) 0605250

(51) Int. Cl. A61K 31/519; A61K 31/357

(52) U.S. Cl. 514/252.16; 514/466

(57) ABSTRACT

The present invention relates to the use of a vasopeptidase inhibitor for the preparation of a medicament for use in the treatment and/or prevention of PAH.

Pulmonary arterial hypertension (PAH) is a fatal disease characterised by intimal lesions, medial hypertrophy and a thickening of the precapillary pulmonary arteries. The increase in the post-charge which results therefrom leads to the development of right ventricular hypertrophy and right cardiac insufficiency. The subjacent pathogenic mechanisms seem to be an increase in vasomotor tonality and chronic transformation of the precapillary resistance vessels.

Treatment of this severe but relatively rare disease has been improved in the last decade by the use of drugs that bring about acute pulmonary vasodilatation, for example prostacyclin derivatives, endothelin receptor antagonists or phosphodiesterase inhibitors. Nevertheless, each of those treatments has disadvantages: prostacyclin agonists have relatively short half-lives and have to be administered by continuous injections, while the others are less effective and/or exhibit liver toxicity (Bull. T. R., Semin. Crit. Care Med., 2005, 26, 425). Consequently, these various therapeutic approaches are often combined but must nevertheless be improved.

Fasidotril, or benzyl (S,S)-2-(2-acetylsulphanylmethyl-3-benzo[1,3]dioxol-5-ylpropionylamino)propionate, is the prototype of a new class of cardiovascular medicaments, often referred to as “vasopeptidase inhibitors”, which combine in the same molecule a powerful inhibitory activity in respect of two metallopeptidases, angiotensin converting enzyme (ACE, EC 3.4.15.1) and neprilysin (EC 3.4.24.11), which are involved in the metabolism of cardiovascular peptide messengers.

It has been demonstrated that vasopeptidase inhibitors can be used in animal models and human clinical trials for arterial hypertension and congestive cardiac events, two relatively common pathological conditions which differ clearly from PAH in terms of pathogenesis, lesions, symptoms and recognised treatments. These medicaments have never been proposed for use in PAH, and there are even experimental reports suggesting that the inhibition of ACE may be ineffective or even detrimental in the animal model of this disease (Ishikawa et al., J. Intern. Cardiol., 1995, 47, 225; Zhou and Lai, J. Appl. Physiol., 1993, 75, 2781).

The present inventors have unexpectedly demonstrated that, on the one hand, vasopeptidase inhibitors used alone are effective in PAH and that, on the other hand, combinations of a vasopeptidase inhibitor and a recognised treatment for PAH are extremely effective in slowing down the development of the disease.

According to a first object, the present invention therefore relates to the use of a vasopeptidase inhibitor, as the only active agent or in combination with a medicament for the treatment of PAH, for the preparation of a medicament for the treatment and/or prevention of PAH.

According to a second object, the present invention relates also to combinations of a vasopeptidase inhibitor and a medicament for the treatment of PAH with a pharmaceutically acceptable vehicle.

According to a further object, the present invention relates also to the use of a combination according to the invention for the preparation of a medicament for the treatment and/or prevention of PAH.

According to another object, the present invention relates also to the above-mentioned methods of therapeutic treatment comprising the administration of a compound or a combination according to the invention to a patient in need thereof.

According to the invention, the combinations enable the two active ingredients to be administered in a separate simultaneous manner or in a manner sequential in time.

Preferably, the combinations according to the invention are presented in a form suitable for administration by the oral route.

Advantageously, combinations demonstrating a synergistic effect are preferred.

According to a preferred aspect, the combinations according to the invention contain the two active ingredients in the same unit dose.

In particular, fasidotril or omapatrilat, and more preferably fasidotril, may be mentioned as the vasopeptidase inhibitor.

In particular, phosphodiesterase inhibitors, more especially PDE-5 inhibitors, such as sildenafil (Viagra®) or tadalafil, preferably sildenafil, may be mentioned as the medicament for the treatment of PAH.

Combinations of fasidotril and sildenafil are especially preferred.

Preferably, the vasopeptidase inhibitor is presented in a form suitable for administration by the oral route. Generally, the vasopeptidase inhibitor is administered from one to three times per day, preferably twice per day, at doses of from 10 to 200 mg, preferably approximately 100 mg twice per day.

Preferably, the PDE-5 inhibitor is presented in a form suitable for administration by the oral route, typically in the form of film-coated tablets. Generally, the PDE-5 inhibitor is administered from one to three times per day, preferably twice per day, at doses of from 10 to 200 mg.

The quantity of each component administered is determined by the treating doctors, taking into account the aetiology and the severity of the disease, the condition and age of the patient, the potency of each component, and other factors.

More generally, the medicaments according to the invention may be presented in forms intended for administration by the parenteral, oral, rectal, permucous or percutaneous route.

They will therefore be presented in the form of injectable suspensions or solutes or multi-dose vials, in the form of uncoated or coated tablets, dragées, capsules, soft gelatin capsules, pills, cachets, powders, rectal capsules or suppositories, solutions or suspensions, for percutaneous use in a polar solvent, for permucous use.

Excipients suitable for such administrations are derivatives of cellulose or of microcristalline cellulose, alkaline-earth carbonates, magnesium phosphate, starches, modified starches, lactose for solid forms.

For rectal use, cocoa butter or polyethylene glycol stearates are the preferred excipients.

For parenteral use, water, aqueous solutes, physiological serum, isotonic solutes are the vehicles most conveniently used.

The dosage may vary within wide limits (0.5 mg to 1000 mg) depending on the therapeutic indication and the route of administration and also on the age and weight of the subject.

“Formulations suitable for oral administration” means formulations which are in a form suitable for being administered orally to a patient. The formulations may be presented in the form of discrete units, such as capsules, cachets or tablets, each containing a predetermined amount of the active ingredient; in the form of a powder or granules; in the form of a solution or a suspension in an aqueous liquid or a non-aqueous liquid; or in the form of an oil-in-water liquid emulsion or a water-in-oil liquid emulsion. The active ingredient may also be presented in the form of a bolus, an electuary or a paste. In solid pharmaceutical forms, the compound useful according to the invention is mixed with at least one inert conventional excipient (or vehicle) such as sodium citrate or dicalcium phosphate or (a) excipients or diluents, such as, for example, starches, lactose, saccharose, glucose, mannitol and silicic acid, (b) binders, such as, for example, carboxymethylcellulose, alginates, polyvinylpyrrolidone, saccharose and acacia, (c) humectants, such as, for example, glycerol, (d) disintegrators, such as, for example, agar-agar, calcium carbonate, potato starch or manioc starch, alginic acid, certain complex silicates and sodium carbonate, (e) dissolution retarders, such as, for example, paraffin, (f) absorption accelerators, such as, for example, quaternary ammonium compounds, (g) wetting agents, such as, for example, cetyl alcohol and glyceryl monostearate, (h) adsorbents, such as, for example, kaolin and bentonite, (i) lubricants, such as, for example, talcum, calcium stearate, magnesium stearate, solid polyethylene glycols, sodium lauryl sulphate, (j) opacifying agents, (k) buffers and agents which release the compound(s) useful according to the invention in a delayed manner in a specific portion of the intestinal tract.

“Formulations suitable for parenteral administration” means formulations which are in a form suitable for being administered to a patient by the parenteral route. The formulations are sterile and include emulsions, suspensions, aqueous and non-aqueous injection solutions, which may contain suspension agents and thickening agents and antioxidants, buffers, bacteriostatics and solutes which render the formulation isotonic, and have a pH adjusted in a suitable manner, with the blood of the desired receiver.

“Formulations suitable for rectal administration” means formulations which are in a form suitable for being administered to a patient by the rectal route. The formulation is preferably in the form of suppositories which can be prepared by mixing the compounds useful according to the present invention with suitable non-irritant vehicles or excipients, such as cocoa butter, polyethylene glycol, or a suppository wax, which are solid at normal temperatures but liquid at body temperature and, thus, molten in the rectum or vaginal cavity, release the active component.

For administration by the percutaneous or permucous route, the formulation may be presented in the form of a topical ointment, balsams, powders, sprays and inhalants, gels (based on water or alcohol), creams, as generally known in the art, or it may be incorporated in a matrix base for application in a patch, which would allow controlled release of the compound via the transdermal barrier.

“Patient” includes both a human and other mammals.

“Pharmaceutical composition” means a composition comprising a compound of formula I and at least one component selected from the group comprising pharmaceutically acceptable transporters, diluents, adjuvants, excipients, or vehicles, such as preserving agents, fillers, disintegrating agents, wetting agents, emulsifying agents, suspension agents, sweetening agents, flavouring agents, perfuming agents, antibacterial agents, antifungal agents, lubricating agents, and dispersing agents, according to the nature of the mode of administration and the dosage forms. Examples of suspension agents include ethoxylated isostearyl alcohols, polyoxyethylene sorbitol and sorbitan esters, microcrystalline cellulose, aluminium metahydroxide, bentonite, agar-agar and tragacanth, or mixtures of those substances. The action of microorganisms can be prevented by various antibacterial and antifungal agents, for example parabens, chlorobutanol, phenol, sorbic acid, and the like. It may also be desirable to include isotonic agents, for example sugars, sodium chloride and the like. The prolonged absorption of the injectable pharmaceutical form can be effected by using absorption-retarding agents, for example aluminium monostearate and gelatin. Examples of suitable transporters, diluents, solvents or vehicles include water, ethanol, polyols, suitable mixtures thereof, vegetable oils (such as olive oil) and injectable organic esters, such as ethyl oleate. Examples of excipients include lactose, milk sugar, sodium citrate, calcium carbonate, dicalcium phosphate. Examples of disintegrating agents include starch, alginic acids and certain complex silicates. Examples of lubricants include magnesium stearate, sodium lauryl sulphate, talcum, and also high-molecular-mass polyethylene glycols.

“Pharmaceutically acceptable” means, within the scope of a valid medical opinion, suitable for use in contact with the cells of humans and lower animals without toxicity, irritation, undue allergic response and the like, commensurate with an advantage/reasonable risk relationship.

The active ingredients can be used in free form or in the form of their pharmaceutically acceptable salts.

The expression “pharmaceutically acceptable salts” refers to the relatively non-toxic, inorganic and organic acid addition salts, and the base addition salts, of the compounds of the present invention. These salts can be prepared in situ during the final isolation and the purification of the compounds. In particular, the acid addition salts can be prepared by reacting the purified compound separately in its purified form with an organic or inorganic acid and by isolating the salt so formed. Examples of acid addition salts include the salts: bromohydrate, chlorohydrate, sulphate, bisulphate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptanate, lactobionate, sulphamates, malonates, salicylates, propionates, methylene bis-b-hydroxynaphthoates, gentisic acid, isethionates, di-p-toluoyl tartrates, methanesulphonates, ethanesulphonates, benzenesulphonates, p-toluene-sulphonates, cyclohexyl sulphamates and quinates laurylsulphonate, and the like. (see, for example, S. M. Berge et al. “Pharmaceutical Salts ” J. Pharm. Sci, 66: p. 1-19 (1977) which is incorporated herein by reference.) The acid addition salts can also be prepared by reacting the purified compound separately in its acidic form with an organic or inorganic base and by isolating the salt so formed. The acid addition salts include amino and metal salts. Suitable metal salts include the salts of sodium, potassium, calcium, barium, zinc, magnesium and aluminium. The salts of sodium and potassium are preferred. Suitable inorganic base addition salts are prepared from metal bases which include sodium hydride, sodium hydroxide, potassium hydroxide, calcium hydroxide, aluminium hydroxide, lithium hydroxide, magnesium hydroxide, zinc hydroxide. Suitable basic amino addition salts are prepared from amines which are sufficiently alkaline to form a stable salt, and preferably include the amines which are often used in medicinal chemistry owing to their low toxicity and their acceptability for medical use: ammonia, ethylenediamine, N-methylglucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, for example lysine and arginine, and dicyclohexylamine, and the like.

The following examples are given by way of non-limiting illustration of the present invention.

### EXAMPLE 1

The long-term efficacy of fasidotril was studied on a recognised PAH model in rats receiving an injection of the toxic alkaloid, monocrotaline (Rosenberg and Rabinovitch, Am. J. Physiol., 1988, 255, 484). After a single injection of the toxin (60 mg/kg, s.c.), the pulmonary arterial endothelium is wounded and the animals develop pulmonary arterial smooth muscle hypertrophy with severe pulmonary hypertension and right ventricular hypertrophy.

A group of 50 rats of the Wistar type weighing approximately 300 g was used, of which 10 were used as controls while 40 received a dose of 60 mg/kg of monocrotaline s.c. The latter were divided into two groups of 20 animals: the first group was fed with a standard laboratory feed while the second group was fed with a feed containing fasidotril at a concentration such that the rats received a dose of approximately 200 mg/kg/day.

After one month, the mortality was 45% in the “monocrotaline only” group but only 20% in the “monocrotaline/fasidotril” group. In addition, the monocrotaline caused a marked right heart hypertrophy with an increase of +140% and +74% in the weight of the right auricle and the right ventricle, respectively, while the corresponding values in the “fasidotril” group were only +57% and +50%.

It therefore seems clear from these data that treatment with fasidotril slowed down the rate of development of the disease in this PAH model by approximately a half.

### EXAMPLE 2

Fasidotril is combined with sildenafil, an inhibitor of phosphodiesterase 5, which was considered active in the PAH model (Schermuly et al., Amer. J. Resp. Crit. Care Med. 2004, 169, 39):

A group of Wistar rats weighing 250-275 g receives monocrotaline by the subcutaneous route (60 mg/kg,s.c.) and is then treated for one month with fasidotrilate (100 mg/kg, b.i.d.), sildenafil (5 mg/kg, b.i.d.), a combination of fasidotrilate and sildenafil, or vehicle (controls). The rats are weighed in the course of the treatments and then sacrificed at the conclusion thereof. The right heart hypertrophy is evaluated, especially by dissection and weighing.

Each of the separate treatments appears to prevent the development of hypertrophy partially compared with the controls but their combination leads to almost total protection. This indicates for the first time the synergistic value of this combination.

1. Method for the treatment and/or the prevention of PAH comprising the administration of a vasopeptidase inhibitor as the only active agent or in combination with a phosphodiesterase inhibitor.

2. Method according to claim 1 such that the vasopeptidase inhibitor is fasidotril.

3. Method according to claim 1 such that the medicament is suitable for administration by the oral route.

4. Method according to claim 1 such that the medicament allows the administration of from 10 to 200 mg of the vasopeptidase inhibitor.

5. Combination of a vasopeptidase inhibitor and a phosphodiesterase inhibitor with a pharmaceutically acceptable vehicle.

6. Combination according to claim 5, such that the vasopeptidase inhibitor is fasidotril.

7. Combination according to claim 5 such that the phosphodiesterase inhibitor is sildenafil.

8. Combination according to claim 5 comprising fasidotril and sildenafil.

9. Combination according to claim 5 enabling the two active ingredients to be administered in a separate simultaneous manner or in a manner sequential in time.

10. Combination according to claim 5 such that it allows the administration of from 10 to 200 mg of the vasopeptidase inhibitor and from 10 to 200 mg of the PDE-5 inhibitor.

11. Method for the treatment and/or prevention of PAH comprising the administration of a combination according to claim 5.
